SEQUENCE LISTING (12 sequences)

SEQ ID NO: 1 - protein, 221 aa, synthetic construct
MARFIAQLLLLASCVAAGQAVTAFLGERVTLTSYWRRVSLGPEIEVSWFKLGPGEEQVLIGRMHHDVIFIEWPFRGFFDIHRSANTFFLVVTAANISHDGNYLCRMKLGETEVTKQEHLSVVKPLTLSVHSERSQFPDFSVLTVTCTVNAFPHPHVQWLMPEGVEPAPTAANGGVMKEKDGSLSVAVDLSLPKPWHLPVTCVGKNDKEEAHGVYVSGYLSQ

SEQ ID NO: 2 - DNA, 666 bp, synthetic construct
  source 1..666: mol_type = other DNA
atggccaggttcatcgctcagctcctcctgttggcctcctgtgtggccgccggccaggctgtcactgcctttctgggggagagggtcacactgacatcttactggaggagagtgagcctgggccccgagatcgaggtgtcttggttcaagctgggacctggagaggagcaggtgctgatcggcaggatgcaccacgacgtgatcttcatcgagtggccctttcggggcttctttgatatccaccgctccgccaacacattctttctggtggtgaccgccgccaacatctctcacgacggcaattatctgtgcaggatgaagctgggcgagacagaggtgaccaagcaggagcacctgagcgtggtgaagccactgaccctgtctgtgcacagcgagcggtcccagttccccgatttttccgtgctgaccgtgacatgtaccgtgaacgcctttccccaccctcacgtgcagtggctgatgcctgagggagtggagccagcaccaacagcagcaaatggcggcgtgatgaaggagaaggacggcagcctgtccgtggccgtggatctgagcctgcctaagccatggcacctgccagtgacctgcgtcggaaagaacgataaggaggaagcacacggagtctatgtcagtggctacctgtcacagtga

SEQ ID NO: 3 - protein, 201 aa, synthetic construct
VTAFLGERVTLTSYWRRVSLGPEIEVSWFKLGPGEEQVLIGRMHHDVIFIEWPFRGFFDIHRSANTFFLVVTAANISHDGNYLCRMKLGETEVTKQEHLSVVKPLTLSVHSERSQFPDFSVLTVTCTVNAFPHPHVQWLMPEGVEPAPTAANGGVMKEKDGSLSVAVDLSLPKPWHLPVTCVGKNDKEEAHGVYVSGYLSQ

SEQ ID NO: 4 - DNA, 606 bp, synthetic construct
  source 1..606: mol_type = other DNA
gtcactgcctttctgggggagagggtcacactgacatcttactggaggagagtgagcctgggccccgagatcgaggtgtcttggttcaagctgggacctggagaggagcaggtgctgatcggcaggatgcaccacgacgtgatcttcatcgagtggccctttcggggcttctttgatatccaccgctccgccaacacattctttctggtggtgaccgccgccaacatctctcacgacggcaattatctgtgcaggatgaagctgggcgagacagaggtgaccaagcaggagcacctgagcgtggtgaagccactgaccctgtctgtgcacagcgagcggtcccagttccccgatttttccgtgctgaccgtgacatgtaccgtgaacgcctttccccaccctcacgtgcagtggctgatgcctgagggagtggagccagcaccaacagcagcaaatggcggcgtgatgaaggagaaggacggcagcctgtccgtggccgtggatctgagcctgcctaagccatggcacctgccagtgacctgcgtcggaaagaacgataaggaggaagcacacggagtctatgtcagtggctacctgtcacagtga

SEQ ID NO: 5 - protein, 18 aa, synthetic construct
MDWTWILFLVAAATRVHS

SEQ ID NO: 6 - DNA, 54 bp, synthetic construct
  source 1..54: mol_type = other DNA
atggattggacttggattctgttcctggtcgccgccgcaactcgggtgcatagc

SEQ ID NO: 7 - protein, 219 aa, synthetic construct
MDWTWILFLVAAATRVHSVTAFLGERVTLTSYWRRVSLGPEIEVSWFKLGPGEEQVLIGRMHHDVIFIEWPFRGFFDIHRSANTFFLVVTAANISHDGNYLCRMKLGETEVTKQEHLSVVKPLTLSVHSERSQFPDFSVLTVTCTVNAFPHPHVQWLMPEGVEPAPTAANGGVMKEKDGSLSVAVDLSLPKPWHLPVTCVGKNDKEEAHGVYVSGYLSQ

SEQ ID NO: 8 - DNA, 660 bp, synthetic construct
  source 1..660: mol_type = other DNA
atggattggacttggattctgttcctggtcgccgccgcaactcgggtgcatagcgtcactgcctttctgggggagagggtcacactgacatcttactggaggagagtgagcctgggccccgagatcgaggtgtcttggttcaagctgggacctggagaggagcaggtgctgatcggcaggatgcaccacgacgtgatcttcatcgagtggccctttcggggcttctttgatatccaccgctccgccaacacattctttctggtggtgaccgccgccaacatctctcacgacggcaattatctgtgcaggatgaagctgggcgagacagaggtgaccaagcaggagcacctgagcgtggtgaagccactgaccctgtctgtgcacagcgagcggtcccagttccccgatttttccgtgctgaccgtgacatgtaccgtgaacgcctttccccaccctcacgtgcagtggctgatgcctgagggagtggagccagcaccaacagcagcaaatggcggcgtgatgaaggagaaggacggcagcctgtccgtggccgtggatctgagcctgcctaagccatggcacctgccagtgacctgcgtcggaaagaacgataaggaggaagcacacggagtctatgtcagtggctacctgtcacagtga

SEQ ID NO: 9 - DNA, 19 bp, synthetic construct
  source 1..19: mol_type = other DNA
cttcatcgagtggcccttt

SEQ ID NO: 10 - DNA, 21 bp, synthetic construct
  source 1..21: mol_type = other DNA
cttcatcctgcacaggtagtt

SEQ ID NO: 11 - DNA, 20 bp, synthetic construct
  source 1..20: mol_type = other DNA
gcctctaacgctgtctgtcc

SEQ ID NO: 12 - DNA, 20 bp, synthetic construct
  source 1..20: mol_type = other DNA
gagaggctcccatccttttc

(19) United States
(12) Patent Application Publication
(10) Pub. No.: US 2025/0127884 A1
(43) Pub. Date: Apr. 24, 2025

(54) Novel Epstein Barr Virus Immunotherapies

(71) Applicant: The Wistar Institute of Anatomy and Biology, Philadelphia, PA (US)

(72) Inventors: David Weiner, Merion, PA (US); Xizhou Zhu, Philadelphia, PA (US)

(21) Appl. No.: 18/688,266

(22) PCT Filed: Aug. 30, 2022
(86) PCT No.: PCT/US2022/075703

§ 371 Date: Feb. 29, 2024

Related U.S. Application Data
(60) Provisional application No. 63/238,498, filed on Aug. 30, 2021.

(51) Int. Cl. A61K 39/245; A61K 39/00; A61P 35/00; C12N 7/00; C12N 15/85

(52) U.S. Cl. CPC A61K 39/245; A61P 35/00; C12N 7/00; C12N 15/85; A61K 2039/53; A61K 2039/572; C12N 2710/16234; C12N 2800/22

(57) ABSTRACT

The present invention generally relates to immunogenic compositions (e.g., vaccines and immunotherapies) that induce an immune response against Epstein Barr Virus (EBV)-associated antigens. The present invention also relates to methods of use thereof for the treatment and prevention of EBV infection, EBV-associated cancer, or a combination thereof. In various embodiments, the EBV antigen comprises BamHI-A rightward frame 1 (BARF1).

### CROSS REFERENCE TO RELATED APPLICATIONS

This application claims priority to U.S. Provisional Application No. 63/238,498, filed Aug. 30, 2021, which is hereby incorporated by reference herein in its entirety.

### BACKGROUND OF THE INVENTION

Epstein Barr Virus (EBV), also known as Human gammaherpesvirus 4 (HHV-4), is highly ubiquitous with more than 95% of the world population infected (de Martel, C., et al., 2020, Lancet Glob Health 8, e180-e190). As the first identified oncogenic virus, EBV is associated with several types of lymphomas and carcinomas, including nasopharyngeal carcinoma (NPC) and EBV-associated gastric carcinoma (EBVaGC) (Young, L. S., et al., 2016, Nat. Rev. Cancer 16, 789-802). NPC and EBVaGC account for more than 92% of all EBV-associated cancers, resulting in approximately 160,000 cases per year globally (Shannon-Lowe, C., and Rickinson, A., 2019, Front. Oncol. 9, 713). The majority of NPC are EBV⁺, exhibiting type III viral latency with the expression of latent membrane proteins (LMP1 and LMP2), EBV nuclear antigen (EBNA1), and EBV Bam HI-A region rightward transcripts (BARTs) (Shannon-Lowe, C., and Rickinson, A., 2019, Front. Oncol. 9, 713). Early and locally advanced cancer responds well to radiation or concurrent chemoradiation therapies. However, treatment for recurrent or metastatic disease is limited and the prognosis is poor (Zhang, Y., et al., 2019, N. Engl. J. Med. 381, 1124-1135). EBVaGC accounts for about 9% of all gastric cancer (GC) and displays a unique molecular signature than other GC subtypes, including showing DNA hypermethylation as well as upregulation of programmed death ligands 1 and 2 (PD-L1/2) (Cancer Genome Atlas Research, N., 2014, Nature 513, 202-209). There is no EBV-targeted therapy approved for NPC and EBVaGC.

Immunotherapy is considered a promising and new approach for treating cancer, for their potential to display acceptable toxicity with designed tumor specificity. Therapies including allogeneic T cell transfer, targeted antibodies, and therapeutic immunizations have been explored in preclinical and clinical studies for NPC and EBVaGC (Smith, C., et al., 2012, Cancer Res. 72, 1116-1125; Fae, D. A., et al., 2016, Cancer Immunol Res 4, 431-440; Turrini, R., et al., 2017, Oncoimmunology 6; Taylor, G. S., et al., 2014, Clin. Cancer Res. 20, 5009-5022). Pembrolizumab, a PD-1 inhibitor, was approved by FDA for recurrent or metastatic NPC (Hsu, C., et al., 2017, J. Clin. Oncol. 35, 4050-4056). However, the overall response rates are limited to 26.3% of patients, with a median progression-free survival (PFS) of 6.5 months. The development of new approaches and immunotherapy targets remain important. Previous studies focused on targeting EBV viral proteins have explored particularly EBNA1, LMP1, and LMP2 antigens as promising therapeutic targets. These remain under study, but alone they have not individually displayed significant impact (Taylor, G. S., et al., 2014, Clin. Cancer Res. 20, 5009-5022; Chia, W. K., et al., 2012, Ann. Oncol. 23, 997-1005). A less studied target, BamHI-A rightward frame 1 (BARF1) is an EBV protein that is found to be highly expressed in NPC and EBVaGC (Decaussin, G., et al., 2000, Cancer Res. 60, 5584-5588; zur Hausen, A., et al., 2000, Cancer Res. 60, 2745-2748). BARF1 is 221 amino acids in length and contains two immunoglobulin-like domains (Blanco, R., and Aguayo, F., 2020, Biology (Basel) 9). It is cleaved after the first 20 amino acids and secreted as a hexamer (sBARF1). BARF1 contains interaction sites that allow it to bind to human macrophage colony-stimulating factor (M-CSF) through its N-terminal domain and human M-CSF-receptor homologous region located in its C-terminal domain. Structural studies have shown that sBARF1 can interfere with monocytes' differentiation through binding to M-CSF as a decoy receptor (Shim, A. H., et al., 2012; Proc. Natl. Acad. Sci. U.S.A 109, 12962-12967). This interaction can reduce the expression of markers for macrophage differentiation such as CD11b, CD14, CD16, and CD169 and inhibit the production of interferon-alpha (IFN-α) by mononuclear cells, which is important for host anti-viral immune response (Blanco, R., and Aguayo, F., 2020, Biology (Basel) 9).

Other oncogenic effects of BARF1 include promoting cell proliferation, inducing cell immortalization, and anti-apoptosis (Sall, A., et al., 2004, Oncogene 23, 4938-4944; Wei, M. X., et al., 1997, Oncogene 14, 3073-3081). Importantly, previous studies have demonstrated BARF1 to be immunogenic, as BARF1-specific antibodies and T cells were detected in some NPC patients (Tanner, J. E., et al., 1997, J. Infect. Dis. 175, 38-46; Martorelli, D., et al., 2008, Int. J. Cancer 123, 1100-1107). Reports also showed that T cells specific to BARF1 epitopes, expanded from patient blood samples ex vivo, are able to kill BARF1⁺ cancer cells (Martorelli, D., et al., 2008, Int. J. Cancer 123, 1100-1107; Kalra, M., et al., Cytotherapy 21, 212-223). Thus, BARF1 appears to be an interesting candidate to be further studied for targeting EBV-associated cancer.

Most immunotherapies described for EBV-associated diseases have focused on adoptive cell transfer (ACT) and therapeutic immunization (Dasari, V., et al., 2019, Expert Rev Vaccines 18, 457-474). For the ACT strategy, EBV-specific cytotoxic T lymphocytes (CTL) were generated in vitro by using EBV-transformed lymphoblastoid cell lines (LCL) as antigen-presenting cells (APCs). Adoptive transfer of CTLs targeting EBNA1, LMP1, and LMP2 has shown some levels of antitumor response in some NPC patients (Fae, D. A., et al., 2016, Cancer Immunol Res 4, 431-440; Comoli, P., et al., 2005, J. Clin. Oncol. 23, 8942-8949). In the therapeutic immunization approach, several different approaches displaying different EBV antigens have been studied in clinical trials. These include autologous dendritic cells pulsed with HLA-restricted epitope peptides from LMP2; recombinant vaccinia virus encoding an EBNA1/LMP2 fusion protein; and a recombinant adenoviral vector expressing the LMP2 antigen (Taylor, G. S., et al., 2014, Clin. Cancer Res. 20, 5009-5022; Lin, C. L., et al., 2002, Cancer Res. 62, 6952-6958; Si, Y., et al., 2016, Chem. Pharm. Bull. (Tokyo) 64, 1118-1123). These studies have shown modest efficacy likely due in part to the limited immunogenicity of LMPs and EBNA1. Additional EBV viral targets might provide additional immune breadth which could improve immunotherapeutic efficacy. BARF1 was found to have induced antigen-specific CTL in some EBV seropositive healthy donors and NPC patients, however, it has not been studied for its potential in therapeutic immunization approaches (Martorelli, D., et al., 2008, Int. J. Cancer 123, 1100-1107). It remains unclear whether immunization with BARF1 can induce immune responses that can affect the progression of EBV⁺ cancer in model systems.

Thus, there is a need in the art for improved vaccines, immunotherapeutic compositions, and methods for the treatment and prevention of EBV infection and EBV-related cancers. This invention satisfies this unmet need.

### SUMMARY OF THE INVENTION

In one embodiment, the present invention relates to an immunogenic composition comprising one or more Epstein Barr Virus (EBV) antigenic polypeptide. In one embodiment, said one or more EBV antigenic polypeptide comprises BamHI-A rightward frame 1 (BARF1) polypeptide. In one embodiment, said one or more EBV antigenic polypeptide comprises a signal peptide. In one embodiment, said signal peptide comprises an IgE signal peptide.

In one embodiment of the immunogenic composition, said BARF1 polypeptide comprises one or more selected from the group consisting of: a polypeptide comprising an amino acid sequence at least 90% identical to SEQ ID NO: 7; a polypeptide fragment comprising an amino acid sequence at least 90% of the full length of SEQ ID NO: 7; and a polypeptide fragment comprising an amino acid sequence at least 90% identical to an amino acid sequence at least 90% of the full length of SEQ ID NO: 7. In one embodiment, said BARF1 polypeptide comprises the amino acid sequence of SEQ ID NO: 7.

In one embodiment, the present invention relates to an immunogenic composition comprising a nucleic acid molecule encoding one or more Epstein Barr Virus (EBV) antigenic polypeptide. In one embodiment, said nucleic acid molecule comprises a nucleotide sequence encoding BamHI-A rightward frame 1 (BARF1) polypeptide. In one embodiment, said nucleic acid molecule comprises a nucleotide sequence encoding a signal peptide. In one embodiment, said nucleic acid molecule comprises a nucleotide sequence encoding an IgE signal peptide.

In one embodiment of the immunogenic composition, said nucleic acid molecule comprises one or more selected from the group consisting of: a nucleic acid molecule comprising a nucleotide sequence at least 90% identical to SEQ ID NO: 8; a nucleic acid fragment comprising a nucleotide sequence at least 90% of the full length of SEQ ID NO: 8; and a nucleic acid fragment comprising a nucleotide sequence at least 90% identical to a nucleotide sequence at least 90% of the full length of SEQ ID NO: 8. In one embodiment, said nucleic acid molecule comprises the nucleotide sequence of SEQ ID NO: 8. In one embodiment, said nucleic acid molecule comprises a codon and RNA optimized nucleotide sequence for expression in mammalian cells.

In one embodiment of the immunogenic composition, said nucleic acid molecule comprises a plasmid expression vector. In one embodiment, said plasmid expression vector comprises a pVax expression vector.

In one embodiment, the present invention relates to a method of administering an immunogenic composition to a subject, comprising administering to the subject one or more selected from the group consisting of: a composition comprising a nucleic acid molecule comprising a nucleotide sequence at least 90% identical to SEQ ID NO: 8; and a composition comprising a polypeptide comprising an amino acid sequence at least 90% identical to SEQ ID NO: 7.

In one embodiment, the present invention relates to a method of inducing an immune response to one or more EBV antigen in a subject, comprising administering to the subject one or more immunogenic composition selected from the group consisting of: a composition comprising a nucleic acid molecule comprising a nucleotide sequence at least 90% identical to SEQ ID NO: 8; and a composition comprising a polypeptide comprising an amino acid sequence at least 90% identical to SEQ ID NO: 7.

In one embodiment, the present invention relates to a method of treating or preventing one or more disease or disorder associated with EBV infection in a subject in need thereof, comprising administering to the subject one or more immunogenic composition selected from the group consisting of: a composition comprising a nucleic acid molecule comprising a nucleotide sequence at least 90% identical to SEQ ID NO: 8; and a composition comprising a polypeptide comprising an amino acid sequence at least 90% identical to SEQ ID NO: 7. In one embodiment, said disease or disorder is cancer. In one embodiment, said cancer comprises one or more selected from the group consisting of: nasopharyngeal carcinoma (NPC), EBV-associated gastric carcinoma (EBVaGC), Hodgkin's lymphoma, Burkitt lymphoma, Diffuse large B cell lymphoma, T cell lymphoma, and NK cell lymphoma.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIG. 1A through FIG. 1C, depicts the design and exemplary results demonstrating in vitro expression of pBARF1. FIG. 1A depicts a schematic representation of native BARF1 protein. FIG. 1B comprises a schematic depiction of pBARF1 plasmid. IgE leader sequence and cloning sites of pVax plasmid vector are indicated. FIG. 1C depicts exemplary Western blot results of pBARF1 expression in supernatant and lysate of transfected 293T cells. Recombinant BARF1 protein and pVax-transfected 293T cells were used as the positive and negative control, respectively.

FIG. 2, comprising FIG. 2A through FIG. 2D, depict exemplary results demonstrating that pBARF1 elicits a high titer of antibody responses. FIG. 2A depicts an outline of the immunogenicity study of pBARF1 in C57BL/6 and BALB/c mice. Sera were collected one week after each immunization, and splenocytes were harvested one week after the final immunization. FIGS. 2B and 2C depict exemplary results showing binding of sera from immunized C57BL/6 (FIG. 2B) and BALB/c (FIG. 2C) mice to recombinant BARF1 protein by ELISA assay. FIG. 2D depicts exemplary results showing the endpoint titer of mice sera, determined by BARF1 binding activity from FIGS. 2B and 2C. n=5 mice/group. Results are representative of two independent experiments. Error bars indicate mean±SEM.

FIG. 3, comprising FIG. 3A through FIG. 3J, depict exemplary results demonstrating that pBARF1 induces potent T cell responses. In FIG. 3A-3J splenocytes from mice immunized with pBARF1 or pVax (outlined in FIG. 2A) were stimulated by native BARF1 peptides. IFN-γ ELISpot assay of stimulated splenocytes was performed for C57BL/6 (FIG. 3A) and BALB/c (FIG. 3B) mice. FIGS. 3C, 3E, 3G, and 3I depict exemplary results showing intracellular staining of IFN-γ, TNF-α, and IL-2 of CD8+ and CD4+ T cells from splenocytes. FIGS. 3D, 3F, 3H, and 3J depicts exemplary pie charts that represent the proportions of each cytokine-producing T cell population in FIGS. 3C, 3E, 3G, and 3I. Significance was determined using the nonparametric Mann-Whitney U test. *p<0.05, **p<0.01. n=5 mice/group. Results are representative of two independent experiments. Error bars indicate mean±SEM.

FIG. 4, comprising FIG. 4A through FIG. 4C, depict exemplary results demonstrating that immunization of pBARF1 improves survival in therapeutic tumor model in C57BL/6 mice. FIG. 4A depicts a study outline for the therapeutic tumor model. The mice were injected with MC38-BARF1 cells and immunized with pBARF1 biweekly starting on day 4. FIG. 4B depicts exemplary results showing tumor volume measurements over time for the mouse study described in FIG. 4A. FIG. 4C depicts exemplary results showing a survival curve for the mouse study described in FIG. 4A. Significance for tumor volume was determined by two-way ANOVA. Significance for survival was determined by the log-rank test. *p<0.05, ***p<0.001. n=9-10 mice/group. Error bars indicate mean±SEM.

FIG. 5, comprising FIG. 5A through FIG. 5D, depict exemplary results demonstrating that pBARF1 completely suppresses cancer progression through CD8+ T cells and maintains long-term tumor control. FIG. 5A depicts a study outline for the therapeutic tumor model. The mice were immunized with one, two, or three doses of pBARF1, two weeks apart. CT26-BARF1 cells were injected one week after the final immunization, and anti-CD4 or anti-CD8 antibodies were given one day before the tumor challenge. FIG. 5B-5C depict exemplary results showing tumor volume measurements (FIG. 5B) and a survival plot (FIG. 5C) of the initial challenge study described in FIG. 5A. n=5 mice/group. Results are representative of two independent experiments. FIG. 5D depicts exemplary results in which mice that received one, two, or three doses of pBARF1 and survived tumor challenge in FIG. 5C were randomized and rechallenged with CT26 or CT26-BARF1 cells, on day 446 post the initial tumor challenge FIG. 5A. The survival curve is shown. n=7-8 mice/group. Significance for tumor volume was determined by two-way ANOVA. Significance for survival was determined by the log-rank test. ***p<0.001, ****p<0.0001. Error bars indicate mean±SEM.

FIG. 6, comprising FIG. 6A through FIG. 6D, depict exemplary results demonstrating that pBARF1-induced immunity mediates rapid clearance of cancer cells. FIG. 6A depicts a study outline for the therapeutic tumor model with IVIS. The mice were immunized with three doses of pBARF1, two weeks apart. CT26-Luc or CT26-BARF1-Luc cells were injected one week after the final immunization. FIGS. 6B-6C depicts exemplary results showing IVIS imaging of tumor-bearing mouse (FIG. 6B) and quantification of the bioluminescence signal (FIG. 6C) captured in FIG. 6B. FIG. 6D depicts an exemplary survival plot of mice with indicated treatment. Significance for total flux was determined by two-way ANOVA. Significance for survival was determined by the log-rank test. **p<0.01, ****p<0.0001. n=5 mice/group. Error bars indicate mean±SEM.

FIG. 7, comprising FIG. 7A through FIG. 7E, depict exemplary results demonstrating the generation and validation of BARF1+ tumor models. FIG. 7A depicts an exemplary workflow of retroviral transduction and single-cell cloning of CT26-BARF1 and MC38-BARF1 cells. FIGS. 7B-7C depict an exemplary flow cytometry analysis on single-cell clones of CT26-BARF1 (FIG. 7B) and MC38-BARF1 (FIG. 7C) cells, after single-cell cloning. FIGS. 7D-7E depict exemplary threshold cycles of BARF1 gene amplification by RT-qPCR in transduced and parental mouse cancer cell lines (FIG. 7D) and human EBV positive (C666-1 and SUN719) and negative (AGS) cancer cell lines (FIG. 7E).

FIG. 8, comprising FIG. 8A through FIG. 8C, depict exemplary results demonstrating that pBARF1 improves survival in the therapeutic tumor model in BALB/c mice. FIG. 8A depicts an exemplary study outline for the therapeutic tumor model. The mice were injected with CT26-BARF1 cells and immunized with pBARF1 biweekly starting on day 4. FIG. 8B depicts exemplary results showing tumor volume measurements of the study described in FIG. 8A. FIG. 8C depicts an exemplary survival curve of the study described in FIG. 8A. Significance for tumor volume was determined by two-way ANOVA. Significance for survival was determined by the log-rank test. *p<0.05, ** p<0.01. n=4 mice/group. Error bars indicate mean±SEM.

FIG. 9, comprising FIG. 9A through FIG. 9C, depict exemplary results demonstrating that single immunization of pBARF1 prevents tumor progression in C57BL/6 mice. FIG. 9A depicts an exemplary study outline for the therapeutic tumor model. The mice were immunized with one dose of pBARF1 and injected with MC38-BARF1 cells one week after immunization. FIG. 9B depicts exemplary results showing tumor volume measurements of the study described in FIG. 9A. FIG. 9C depicts an exemplary survival curve of the study described in FIG. 9A. Significance for tumor volume was determined by two-way ANOVA. Significance for survival was determined by the log-rank test. ****p<0.0001. n=10 mice/group. Error bars indicate mean±SEM.

### DETAILED DESCRIPTION

In one embodiment, the present invention relates to an immunogenic composition comprising one or more EBV antigen. In one embodiment, the present invention relates to an immunogenic composition comprising a nucleic acid molecule encoding one or more EBV antigen. In one embodiment, said EBV antigen comprises BARF1. In one embodiment, the nucleic acid molecule is codon optimized. In one embodiment, the nucleic acid molecule is RNA optimized. In one embodiment, the nucleic acid molecule further comprises a nucleotide sequence encoding a signal peptide.

In one embodiment, the present invention relates to methods of inducing an immune response in a subject against one or more EBV antigen. In one embodiment, the inventions comprise methods of treating or preventing one or more disease or disorder associated with EBV infection in a subject in need thereof. In one embodiment, the disease or disorder comprises cancer.

The present invention is based, in part, upon the discovery that the EBV antigen, BamHI-A rightward frame 1 (BARF1), induces robust and specific immune responses when expressed in vivo. Both the generation of specific antibodies and the induction of cytotoxic T cells are described herein.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “adjuvant” as used herein is defined as any molecule to enhance an antigen-specific adaptive immune response.

The term “antigen” or “antigenic” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequence or a partial nucleotide sequence encoding a protein that elicits an adaptive immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “antigenic polypeptide” encompasses immunogenic full-length proteins or fragments of the immunogenic protein (i.e. an immunogenic polypeptide fragment that induces or is capable of inducing an immune response to one or more pathogenic species or cancerous tissue).

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of a subject or mammal to whom the nucleic acid is administered.

“Complement” or “complementary” as used herein refers to Watson-Crick (e.g., A-T/U and C-G) or Hoogsteen base pairing between nucleotides or nucleotide analogs of nucleic acid molecules.

A “disease” is a state of health of a subject wherein the subject cannot maintain homeostasis, and wherein if the disease is not ameliorated then the subject's health continues to deteriorate.

In contrast, a “disorder” in a subject is a state of health in which the subject is able to maintain homeostasis, but in which the subject's state of health is less favorable than it would be in the absence of the disorder. Left untreated, a disorder does not necessarily cause one decrease in the subject's state of health.

The terms “effective amount” and “pharmaceutically effective amount” refer to a sufficient amount of an agent to provide the desired biological result. That result can be reduction and/or alleviation of the signs, symptoms, or causes of a disease or disorder (e.g., prostate cancer), or any other desired alteration of a biological system. An appropriate effective amount in any individual case may be determined by one of ordinary skill in the art using routine experimentation. An “effective amount” or “therapeutically effective amount” of a compound is that amount of compound, which is sufficient to provide a beneficial effect to the subject to which the compound is administered.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “expressible form” refers to genetic constructs that contain the necessary regulatory elements operable linked to a coding sequence that encodes a protein such that when present in the cell of the subject, the coding sequence will be expressed.

As used herein, the term “genetic construct” refers to the DNA or RNA molecules that comprise a nucleotide sequence which encodes a protein. The coding sequence includes initiation and termination signals operably linked to regulatory elements including a promoter and polyadenylation signal capable of directing expression in the cells of the subject to whom the nucleic acid molecule is administered.

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Immune response,” as the term is used herein, means a process involving the activation and/or induction of an effector function in, by way of non-limiting examples, a T cell, B cell, natural killer (NK) cell, and/or an antigen-presenting cell (APC). Thus, an immune response, as would be understood by the skilled artisan, includes, but is not limited to, any detectable antigen-specific activation and/or induction of a helper T cell or cytotoxic T cell activity or response, production of antibodies, antigen presenting cell activity or infiltration, macrophage activity or infiltration, neutrophil activity or infiltration, and the like.

The term “immunogen” or “immunogenic” as used herein, is intended to denote a substance of matter, which is capable of inducing an adaptive immune response in an individual, where said adaptive immune response is capable of inducing an immune response which significantly engages pathogenic agents, which share immunological features with the immunogen. “Immunogen” refers to any substance introduced into the body in order to generate an immune response. That substance can a physical molecule, such as a protein, or can be encoded by a vector, such as DNA, mRNA, or a virus.

As used herein, the terms “immunogenic fragment” refer to a fragment of an antigen or a nucleic acid sequence encoding an antigen that, when administered to a subject, provides an increased immune response. Fragments are generally 10 or more amino acids or nucleic acids in length. “Fragment” may mean a polypeptide fragment of an antigen that is capable of eliciting an immune response in a subject. A fragment of an antigen may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antigen, excluding any heterologous signal peptide added. The fragment may comprise a fragment of a polypeptide that is 95% or more, 96% or more, 97% or more, 98% or more or 99% or more identical to the antigen and additionally comprise an N terminal methionine or heterologous signal peptide which is not included when calculating percent identity.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions. Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control.

A “peptide,” “protein,” or “polypeptide” as used herein can mean a linked sequence of amino acids and can be natural, synthetic, or a modification or combination of natural and synthetic.

As used herein, “pharmaceutically acceptable” means that drugs, medicaments or inert ingredients which the term describes are suitable for use in contact with the tissues of humans and lower animals without undue toxicity, incompatibility, instability, irritation, allergic response, and the like, commensurate with a reasonable benefit/risk ratio.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use.

“Promoter” as used herein means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, S V40 early promoter or SV40 late promoter and the CMV IE promoter.

“Signal peptide” as used herein refers to an amino acid sequence that can be linked at the amino terminus of an antigenic protein set forth herein. Signal peptides typically direct localization of a protein. Signal peptides may facilitate secretion of the protein from the cell in which it is produced. Signal peptides are often cleaved from the remainder of the protein, often referred to as the mature protein, upon secretion from the cell. Signal peptides may be linked at the N terminus of the protein.

The terms “subject,” “patient,” “individual,” and the like are used interchangeably herein, and refer to any animal, or cells thereof whether in vitro or in situ, amenable to the methods described herein. In some non-limiting embodiments, the patient, subject or individual is a mammal, bird, poultry, cattle, pig, horse, sheep, ferret, primate, dog, cat, guinea pig, rabbit, bat, or human.

“Substantially identical,” as used herein, refers to two or more sequences which have a percentage of sequential units which are the same when compared and aligned for maximum correspondence over a comparison window, or designated region as measured using a comparison algorithm or by manual alignment and visual inspection. Two nucleotide sequences or two amino acid sequences are considered substantially identical if they are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1170, 1260, 1350, 1440, 1530, 1620, 1710, 1800, 1890, 1980, 2070 or more nucleotides or amino acids.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “therapeutic” as used herein means a treatment and/or prophylaxis. A therapeutic effect is obtained by suppression, diminution, remission, prevention, or eradication of at least one sign or symptom of a disease or disorder.

To “treat” a disease as the term is used herein, means to reduce the frequency or severity of at least one sign or symptom of a disease or disorder experienced by a subject.

“Variant” used herein with refers to a nucleic acid or polypeptide that has substantial functional similarly to one or more nucleic acid or polypeptide of the present invention. With respect to a nucleic acid, a variant refers to (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto. With respect to a polypeptide, a variant refers to (i) a portion or fragment that is identical to a portion of a referenced amino acid sequence, (ii) (i) a portion or fragment that is substantially identical to a portion of a referenced amino acid sequence, and (iii) a full-length polypeptide that is substantially identical to the full length of a referenced amino acid sequence.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

### Compositions

The present invention generally relates to antigenic polypeptides, variants and fragments thereof, or nucleic acid molecules encoding antigenic polypeptides, variants and fragments thereof, capable of inducing an immune response to Epstein Barr Virus (EBV) for the treatment and prevention of EBV associated diseases and disorders.

### Antigenic Polypeptides

In one embodiment, the present invention relates to a composition comprising one or more Epstein Barr Virus (EBV) antigenic polypeptide. In one embodiment, the EBV antigenic polypeptide comprises one or more EBV viral protein that is expressed at greater than normal levels in one or more cancer tissue. In one embodiment, the cancer tissue comprises one or more selected from the group consisting of: nasopharyngeal carcinoma (NPC), EBV-associated gastric carcinoma (EBVaGC), Hodgkin's lymphoma, Burkitt lymphoma, Diffuse large B cell lymphoma, T cell lymphoma, and NK cell lymphoma.

In one embodiment, the EBV viral protein comprises BamHI-A rightward frame 1 (BARF1). In one embodiment, said BARF1 is viral BARF1. In one embodiment, said viral BARF1 is EBV BARF1.

In one embodiment, the EBV viral protein comprises a polypeptide comprising an amino acid sequence with a sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% to one or more selected from the group consisting of SEQ ID NO: 1, SEQ ID NO: 3 and SEQ ID NO: 7. In one embodiment, the EBV viral protein comprises a polypeptide fragment with a length of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% the length of one or more selected from the group consisting of SEQ ID NO: 1, SEQ ID NO: 3 and SEQ ID NO: 7.

In one embodiment, the EBV viral protein comprises a polypeptide comprising an amino acid sequence with (a) a sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% to one or more selected from the group consisting of SEQ ID NO: 1, SEQ ID NO: 3 and SEQ ID NO: 7; and (b) a length of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% the length of SEQ ID NO: one or more selected from the group consisting of SEQ ID NO: 1, SEQ ID NO: 3 and SEQ ID NO: 7.

In one embodiment, the EBV viral protein comprises an amino acid sequence of one or more selected from the group consisting of SEQ ID NO: 1, SEQ ID NO: 3 and SEQ ID NO: 7.

In one embodiment, the composition further comprises one or more additional EBV viral protein. In one embodiment, the one or more additional EBV viral protein is selected from the group consisting of: latent membrane protein 1 (LMP1), latent membrane protein 2 (LMP2), and EBV nuclear antigen (EBNA1) In one embodiment, the composition further comprises an adjuvant.

A polypeptide of the invention may be synthesized by conventional techniques. For example, the polypeptides may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a peptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a polypeptide the invention conjugated with other molecules may be prepared by fusing, through recombinant techniques, the N-terminal or C-terminal of the polypeptide, and the sequence of a selected protein or selectable marker with a desired biological function. The resultant fusion proteins contain the fusion protein fused to the selected protein or marker protein as described herein. Examples of proteins which may be used to prepare fusion proteins include immunoglobulins, glutathione-S-transferase (GST), hemagglutinin (HA), and truncated myc.

Polypeptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The polypeptides of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benzenesulfonic acid, and toluenesulfonic acids.

### Nucleic Acids

In one embodiment, the composition of the present invention comprises at least one nucleic acid molecule having an open reading frame encoding at least one EBV antigenic polypeptide. In one embodiment, the nucleic acid molecule is RNA and codon optimized. In one embodiment, the nucleic acid molecule comprises a nucleic acid sequence that is RNA and codon optimized for expression in cells of a mammalian subject. In one embodiment, the nucleic acid molecule comprises a nucleic acid sequence that is RNA and codon optimized for expression in cells of primates. In one embodiment, the nucleic acid molecule comprises a nucleic acid sequence that is RNA and codon optimized for expression in cells of humans.

In one embodiment, the nucleic acid molecule encodes one or more EBV viral protein that is expressed at greater than normal levels in one or more cancer tissue (EBV+). In one embodiment, the cancer tissue comprises one or more selected from the group consisting of: nasopharyngeal carcinoma (NPC), EBV-associated gastric carcinoma (EBVaGC), Hodgkin's lymphoma, Burkitt lymphoma, Diffuse large B cell lymphoma, T cell lymphoma, and NK cell lymphoma.

In one embodiment, the nucleic acid molecule encodes an EBV viral protein comprising BamHI-A rightward frame 1 (BARF1). In one embodiment, said BARF1 is viral BARF1. In one embodiment, said viral BARF1 is EBV BARF1.

In one embodiment, the nucleic acid molecule encoding an EBV viral protein comprises a nucleotide sequence with a sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% to one or more selected from the group consisting of SEQ ID NO: 2, SEQ ID NO: 4 and SEQ ID NO: 8. In one embodiment, the nucleic acid molecule encoding an EBV viral protein comprises a nucleotide sequence with a length of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% the length of one or more selected from the group consisting of SEQ ID NO: 2, SEQ ID NO: 4 and SEQ ID NO: 8.

In one embodiment, the nucleic acid molecule encoding an EBV viral protein comprises a nucleotide sequence with (a) a sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% to one or more selected from the group consisting of SEQ ID NO: 2, SEQ ID NO: 4 and SEQ ID NO: 8; and (b) a length of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% the length of one or more selected from the group consisting of SEQ ID NO: 2, SEQ ID NO: 4 and SEQ ID NO: 8.

In one embodiment, the nucleic acid molecule comprises one or more nucleotide sequence selected from the group consisting of SEQ ID NO: 2, SEQ ID NO: 4 and SEQ ID NO: 8.

In one embodiment, the nucleic acid molecule further encodes one or more additional EBV viral protein. In one embodiment, the one or more additional EBV viral protein is selected from the group consisting of: latent membrane protein 1 (LMP1), latent membrane protein 2 (LMP2), and EBV nuclear antigen (EBNA1). In one embodiment, the nucleic acid molecule further encodes one or more adjuvant. In one embodiment, the composition of the invention further comprises one or more additional nucleic acid molecule encoding one or more additional EBV viral protein, as described. In one embodiment, the composition of the invention further comprises one or more additional nucleic acid molecule encoding one or more adjuvant.

The form of the nucleic acid molecule used in an immunogenic composition of the disclosure can be any suitable for stimulating an immune response against EBV when administered to a subject. For example, the nucleic acid can be in the form of “naked DNA” or it can be incorporated in an expression vector. A description of suitable nucleic acids is presented below. Nucleic acids that are most immunogenic in a subject can be determined by preparing several of the below listed nucleic acids (e.g., those that encode the whole antigen, variants or peptide fragments thereof), administering to the subject (or a series of genetically similar such subjects) such nucleic acids in a composition (e.g., as naked nucleic acid or in an expression vector in a suitable carrier), and analyzing the subject(s) for the stimulation of an immune response. Those nucleic acids that induce the desired response can then be selected.

Nucleic acid molecules utilized in the present disclosure as an antigenic agent may be in the form of RNA or in the form of DNA (e.g., cDNA, genomic DNA, and synthetic DNA). The DNA may be double-stranded or single-stranded, and if single-stranded may be the coding (sense) strand or non-coding (anti-sense) strand.

The nucleic acid molecules provide herein, including their regions and/or parts may be RNA and codon optimized. Codon optimization methods are known in the art and may be useful in efforts to achieve one or more of several goals, including but not limited to: to match codon frequencies in target and host organisms to ensure proper folding, bias and/or GC content to increase mRNA stability or reduce secondary structures; to minimize tandem repeat codons or base runs that may impair gene construction or expression; to customize transcriptional and translational control regions; to introduce or remove protein trafficking sequences; to remove or add post translation modification sites in encoded proteins (e.g. glycosylation sites); to add, remove or shuffle protein domains; to insert or delete restriction sites; to modify ribosome binding sites and mRNA degradation sites; to adjust translational rates to allow the various domains of the protein to fold properly, or to reduce or eliminate problematic secondary structures within the polynucleotide. Codon optimization tools, algorithms and services are known in the art.

The nucleic acid molecule sequence encoding a polypeptide can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

### Vectors

The invention also provides for the use of expression vectors to stimulate an immune response against one or more EBV antigenic polypeptide, variant or an immunogenic fragment thereof. In a typical application of this technique, a nucleic acid encoding one or more peptide or protein antigens of EBV is incorporated into a vector that allows expression of the antigen(s) in a host cell (e.g., a cell inside a subject or administered to a subject). The nucleic acid encoding the antigen(s) is generally under the operational control of other sequences contained within the vector such as a promoter sequences (e.g., tissue specific, constitutively active, or inducible) or enhancer sequences. The antigen(s) encoded by the vector are expressed when the vector is introduced into a host cell in a subject. After expression, the antigen(s) can associate with an MHC molecule for presentation to immune system cells such as T lymphocytes, thus stimulating an immune response. See. e.g., Corr et al., J. Exp. Med. 184:1555, 1996.

Vectors for use in the invention can be any capable of expressing an encoded antigen(s) in a subject. For example, vectors derived from bacterial plasmids and viruses may be used. Representative viral vectors include retroviral, adenoviral, and adeno-associated viral vectors. See. e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996. The one or more vectors can contain an origin of replication. The vectors can be a plasmid, bacteriophage, bacterial artificial chromosome or yeast artificial chromosome. The vectors can be either a self-replication extra chromosomal vector, or a vector which integrates into a host genome.

In one embodiment, the disclosure provides a vector comprising a regulatory element operable in a eukaryotic cell (e.g., a mammalian cell such as a human cell) operably linked to a nucleic acid described herein. In some embodiments, the vector comprises a DNA or DNA plasmid vector.

In one embodiment, the vector comprises a nucleotide sequence with a sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% to one or more selected from the group consisting of SEQ ID NO: 2, SEQ ID NO: 4 and SEQ ID NO: 8. In one embodiment, the vector comprises a nucleotide sequence with a length of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% the length of one or more selected from the group consisting of SEQ ID NO: 2, SEQ ID NO: 4 and SEQ ID NO: 8.

In one embodiment, the vector comprises a nucleotide sequence with (a) a sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% to one or more selected from the group consisting of SEQ ID NO: 2, SEQ ID NO: 4 and SEQ ID NO: 8; and (b) a length of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% the length of one or more selected from the group consisting of SEQ ID NO: 2, SEQ ID NO: 4 and SEQ ID NO: 8.

In one embodiment, the vector comprises one or more nucleotide sequence selected from the group consisting of SEQ ID NO: 2, SEQ ID NO: 4 and SEQ ID NO: 8.

In some embodiments it can be preferred that the vectors used for in vivo applications are attenuated to prevent the vector from amplifying in the subject. For example, if plasmid vectors are used, preferably they will lack an origin of replication that functions in the subject so as to enhance safety for in vivo use in the subject. If viral vectors are used, preferably they are attenuated or replication-defective in the subject, again, so as to enhance safety for in vivo use in the subject.

In one embodiment, the vector comprises a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered. The plasmid may also comprise a mammalian origin of replication in order to maintain the plasmid extrachromosomally and produce multiple copies of the plasmid in a cell.

### Signal Peptides

In some embodiments, the antigenic polypeptide of the present invention comprises a signal peptide. In some embodiments, the nucleic acid molecule of the present invention comprises a nucleotide sequence encoding a signal peptide. Signal peptides, commonly comprising the N-terminal 15-60 amino acids of proteins, are typically needed for the translocation across the membrane on the secretory pathway and, thus, universally control the entry of most proteins both in eukaryotes and prokaryotes to the secretory pathway. Signal peptides generally include three regions: an N-terminal region of differing length, which usually comprises positively charged amino acids; a hydrophobic region; and a short carboxy-terminal peptide region. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by a ER-resident signal peptidase of the host cell, or they remain uncleaved and function as a membrane anchor. A signal peptide may also facilitate the targeting of the protein to the cell membrane. The signal peptide, however, is not responsible for the final destination of the mature protein. Secretory proteins devoid of additional address tags in their sequence are by default secreted to the external environment. During recent years, a more advanced view of signal peptides has evolved, showing that the functions and immunodominance of certain signal peptides are much more versatile than previously anticipated.

In some embodiments, the signal peptide fused to the antigenic polypeptide is an artificial signal peptide. For example, in some embodiments, an artificial signal peptide fused to the antigenic polypeptide is obtained from an immunoglobulin protein, e.g., an IgE signal peptide or an IgG signal peptide. In one embodiment, the signal peptide is an IgE signal peptide. In one embodiment, the signal peptide comprises an amino acid sequence of SEQ ID NO: 5. In one embodiment, the signal peptide is encoded by a nucleic acid comprising the nucleotide sequence of SEQ ID NO: 6. In some embodiments, a signal peptide fused to the antigenic polypeptide is an Ig heavy chain epsilon-1 signal peptide (IgE HC SP). In some embodiments, a signal peptide fused to the antigenic polypeptide is an IgGk chain V-III region HAH signal peptide (IgGk SP). In some embodiments, the signal peptide is selected from: Japanese encephalitis PRM signal sequence, VSVg protein signal sequence and Japanese encephalitis JEV signal sequence.

The examples disclosed herein are not meant to be limiting and any signal peptide that is known in the art to facilitate targeting of a protein to ER for processing and/or targeting of a protein to the cell membrane may be used in accordance with the present disclosure. A signal peptide is typically cleaved from the nascent polypeptide at the cleavage junction during ER processing. Therefore, in some embodiments, the mature antigenic polypeptide of the present disclosure after cellular processing does not comprise a signal peptide.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

### Methods

The methods of the present invention generally relate to a administering an immunogenic composition comprising an antigenic polypeptide or a nucleic acid molecule encoding an antigenic polypeptide, as described above, to a subject to induce an immune response against EBV to treat or prevent diseases or disorders associated with EBV infection.

### Administration and Immune Induction

In one embodiment, the present invention relates to a method of administering an immunogenic composition to a subject. In one embodiment, the method comprises administering to the subject a composition comprising one or more polypeptide, variant or fragment thereof of the present invention, as described above. In one embodiment, the method comprises administering to the subject a composition comprising one or more nucleic acid molecule, variant or fragment thereof of the present invention, as described above.

In one embodiment, the present invention relates to a method of inducing an immune response to one or more EBV antigen in a subject. In one embodiment, the method comprises administering to the subject an effective amount of a composition comprising one or more polypeptide, variant or fragment thereof of the present invention, as described above. In one embodiment, the method comprises administering to the subject an effective amount of a composition comprising one or more nucleic acid molecule, variant or fragment thereof of the present invention, as described above.

Administration of the compositions of the disclosure in a method of treatment can be achieved in a number of different ways, using methods known in the art. In one embodiment, the method of the disclosure comprises systemic administration of the subject, including for example enteral or parenteral administration. In certain embodiments, the method comprises intradermal delivery of the composition. In another embodiment, the method comprises intravenous delivery of the composition. In some embodiments, the method comprises intramuscular delivery of the composition. In one embodiment, the method comprises subcutaneous delivery of the composition. In one embodiment, the method comprises inhalation of the composition. In one embodiment, the method comprises intranasal delivery of the composition.

It will be appreciated that the composition of the disclosure may be administered to a subject either alone, or in conjunction with a second therapeutic agent. In one embodiment, the second therapeutic agent comprises an adjuvant.

Typically, dosages which may be administered in a method of the disclosure to a mammal, for example a human, range in amount from 0.01 g to about 50 mg per kilogram of body weight of the mammal, while the precise dosage administered will vary depending upon any number of factors, including but not limited to, the type of mammal and type of disease state being treated, the age of the mammal and the route of administration. In some embodiments, the dosage of the compound will vary from about 0.1 g to about 10 mg per kilogram of body weight of the mammal. In some embodiments, the dosage will vary from about 1 g to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In certain embodiments, administration of an immunogenic composition of the present disclosure may be performed by single administration or boosted by multiple administrations.

### Treatment and Prevention

In one embodiment, the present invention relates to a method of treating or preventing one or more disease or disorder associated with EBV infection in a subject in need thereof. In one embodiment, the method comprises administering to the subject an effective amount of a composition comprising one or more polypeptide, variant or fragment thereof of the present invention, as described above.

In one embodiment, the method comprises administering BamHI-A rightward frame 1 (BARF1). In one embodiment, said BARF1 is viral BARF1. In one embodiment, said viral BARF1 is EBV BARF1.

In one embodiment, the method comprises administering a polypeptide comprising an amino acid sequence with a sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% to one or more selected from the group consisting of SEQ ID NO: 1, SEQ ID NO: 3 and SEQ ID NO: 7. In one embodiment the method comprises administering a polypeptide fragment with a length of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% the length of one or more selected from the group consisting of SEQ ID NO: 1, SEQ ID NO: 3 and SEQ ID NO: 7.

In one embodiment, the method comprises administering a polypeptide comprising an amino acid sequence with (a) a sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% to one or more selected from the group consisting of SEQ ID NO: 1, SEQ ID NO: 3 and SEQ ID NO: 7; and (b) a length of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% the length of one or more selected from the group consisting of SEQ ID NO: 1, SEQ ID NO: 3 and SEQ ID NO: 7.

In one embodiment, the method comprises administering a polypeptide comprising an amino acid sequence of one or more selected from the group consisting of SEQ ID NO: 1, SEQ ID NO: 3 and SEQ ID NO: 7.

In one embodiment, the method further comprises administering one or more additional EBV viral protein. In one embodiment, the one or more additional EBV viral protein is selected from the group consisting of: latent membrane protein 1 (LMP1), latent membrane protein 2 (LMP2), and EBV nuclear antigen (EBNA1). In one embodiment, the method further comprises administering one or more adjuvant.

In one embodiment, the method comprises administering to the subject an effective amount of a composition comprising one or more nucleic acid molecule, variant or fragment thereof of the present invention, as described above.

In one embodiment, the method comprises administering a nucleic acid molecule encoding BamHI-A rightward frame 1 (BARF1). In one embodiment, the method comprises administering a nucleic acid molecule encoding viral BARF1. In one embodiment, the method comprises administering a nucleic acid molecule encoding EBV BARF1.

In one embodiment, the method comprises administering a nucleic acid molecule comprising a nucleotide sequence with a sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% to one or more selected from the group consisting of SEQ ID NO: 2, SEQ ID NO: 4 and SEQ ID NO: 8. In one embodiment, the method comprises administering a nucleic acid molecule comprising a nucleotide sequence with a length of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% the length of one or more selected from the group consisting of SEQ ID NO: 2, SEQ ID NO: 4 and SEQ ID NO: 8.

In one embodiment, the method comprises administering a nucleic acid molecule comprising a nucleotide sequence with (a) a sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% to one or more selected from the group consisting of SEQ ID NO: 2, SEQ ID NO: 4 and SEQ ID NO: 8; and (b) a length of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% the length of one or more selected from the group consisting of SEQ ID NO: 2, SEQ ID NO: 4 and SEQ ID NO: 8.

In one embodiment, the method further comprises administering a nucleic acid molecule encoding one or more additional EBV viral protein. In one embodiment, the one or more additional EBV viral protein is selected from the group consisting of: latent membrane protein 1 (LMP1), latent membrane protein 2 (LMP2), and EBV nuclear antigen (EBNA1). In one embodiment, the method further comprises administering a nucleic acid molecule encoding one or more adjuvant. In one embodiment, the method further comprises administering one or more additional nucleic acid molecule encoding one or more additional EBV viral protein, as described. In one embodiment, the method further comprises administering one or more additional nucleic acid molecule encoding one or more adjuvant

In one embodiment, the disease or disorder comprises infectious mononucleosis. In one embodiment, the disease or disorder comprises cancer. In one embodiment, said cancer comprises an EBV+ cancer. In one embodiment, said EBV+ cancer comprises one or more selected from the group consisting of: nasopharyngeal carcinoma (NPC), EBV-associated gastric carcinoma (EBVaGC), Hodgkin's lymphoma, Burkitt lymphoma, Diffuse large B cell lymphoma, T cell lymphoma, and NK cell lymphoma.

The therapeutic and prophylactic methods of the disclosure further encompass the use of pharmaceutical compositions encoding an antigen, described herein to practice the methods of the disclosure. The pharmaceutical compositions useful for practicing the invention may be administered to deliver a dose of between 1 ng/kg/day and 100 mg/kg/day. In one embodiment, the invention envisions administration of a dose which results in a concentration of the compound of the present disclosure from 10 nM and 10 μM in a mammal.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs.

Pharmaceutical compositions that are useful in the methods of the disclosure may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the disclosure may further comprise one or more additional pharmaceutically active agents.

Controlled- or sustained-release formulations of a pharmaceutical composition of the disclosure may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the disclosure may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the disclosure are known in the art and described, for example in Remington's Pharmaceutical Sciences, 1985, Genaro, ed., Mack Publishing Co., Easton, PA), which is incorporated herein by reference.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: DNA Immunotherapy Targeting BARF1 Induces Potent Antitumor Responses Against Epstein-Barr Virus-Associated Carcinomas

Here, an optimized immunogen encoding the EBV antigen BARF1 (pBARF1) was developed as a synthetic DNA plasmid. It was observed that immunization with pBARF1 induced both CD4⁺ and CD8⁺ T cell responses in both C57BL/6 and BALB/c mice. Potent serological responses were induced irrespective of animal strain. As there is no simple model to study immune responses targeting EBV⁺ tumors in mice, next two BARF1⁺ carcinoma models were established to allow immune impact studies in both C57BL/6 and BALB/c mice. Using these models, it was observed that immunization of pBARF1 significantly improved animal survival in the therapeutic setting. In the pre-challenge immunization model, one dose of pBARF1 was able to completely suppress tumor growth. It was demonstrated that this tumor impact was associated with the induction of CD8⁺ T cell immunity. Finally, using an in vivo imaging system (IVIS), it was observed that pBARF1-induced immunity cleared tumor cells as early as two days post-challenge. These data suggest that pBARF1 may be important as a possible therapeutic target for EBV immune therapy and that further study is warranted.

### Results

Design and In Vitro Expression of pBARF1

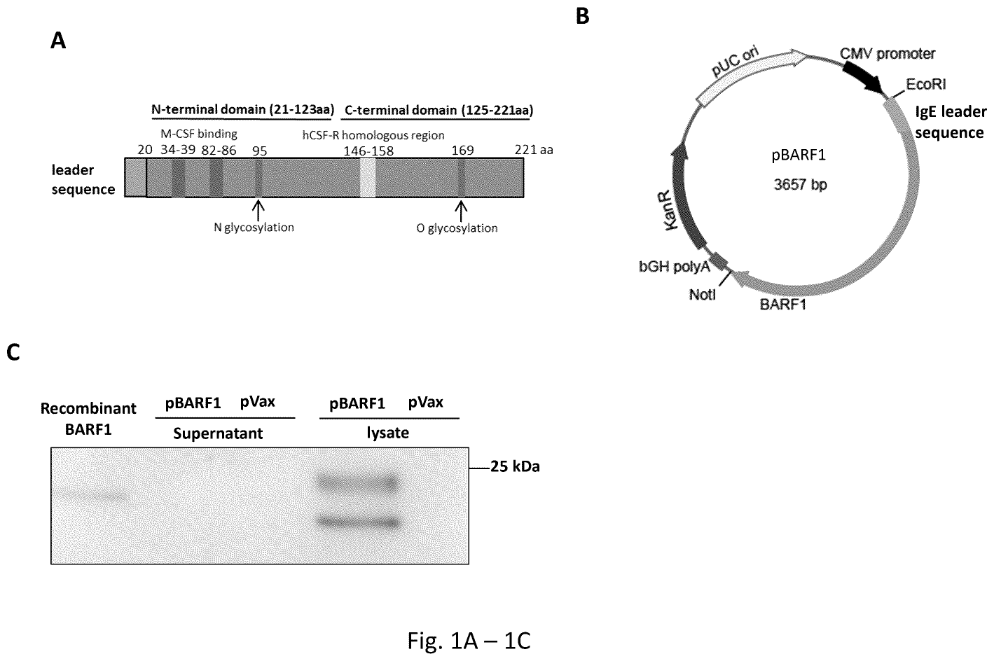

Native BARF1 protein consists of 221 amino acids (FIG. 1A). It contains an N glycosylation on asparagine 95 (Asn95) which is important for protein folding and secretion and an O glycosylation on threonine 169 (Thr169). After cleavage of the signal peptide (1-20 residue), sBARF1 (21-221 residue) is secreted as a hexamer that is complexed by three dimers in two layers. The sBARF1 was shown to interfere with macrophage differentiation through its binding directly to M-CSF. Here, the native BARF1 gene was studied, which is 100% conserved between EBV strains B95.8, GD1, and AG876, in the pBARF1 plasmid design. The DNA plasmid was synthesized by replacing the BARF1 native signal peptide sequence with an IgE leader sequence for enhanced expression (Perales-Puchalt, A., et al., 2019, Mol. Ther. 27, 314-325; Xu, Z., et al., 2020, Advanced Science). The DNA sequence was codon and RNA optimized and cloned into a pVax expression vector (FIG. 1B). After the development of the pBARF1 plasmid, the construct was transfected into 293T cells to confirm its expression in vitro. BARF1 protein was observed primarily in the cell lysate, and the double bands suggested BARF1 detection both pre- and post-cleavage of the IgE signal peptide (FIG. 1C).

pBARF1 Elicits High Titers of Antibody Responses

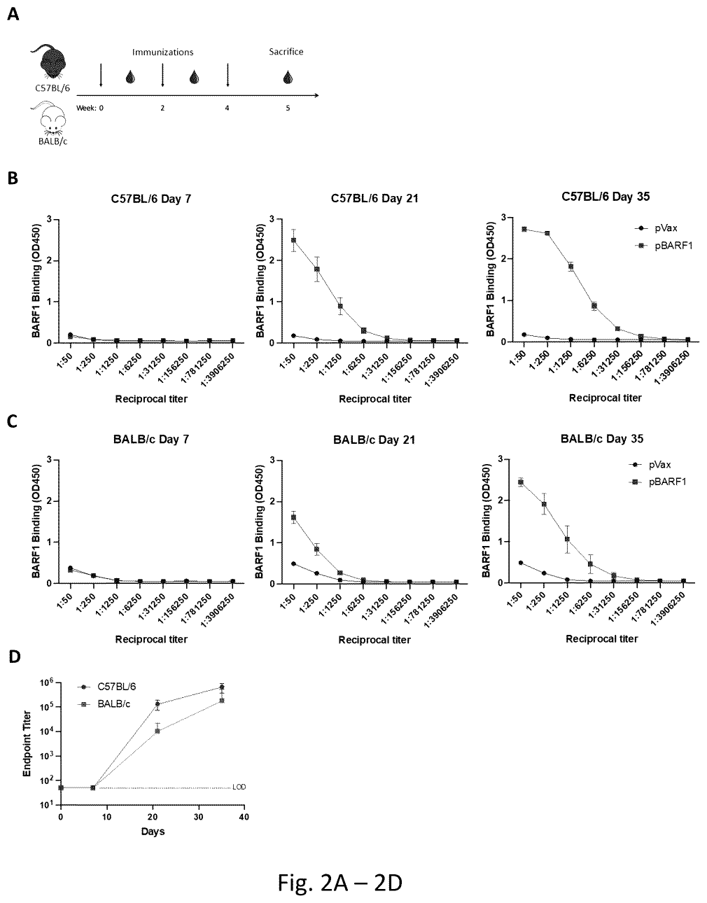

To determine the immunogenicity of the synthetic pBARF1, both C57BL/6 and BALB/c mice were immunized with 25 g of pBARF1 or pVax control three times at two-week intervals (FIG. 2A). Since BARF1 is expressed on the cell surface and mostly secreted (Houali, K., et al., 2007, Clin. Cancer Res. 13, 4993-5000), the antibody response induced by pBARF1 was examined. Mouse sera was collected one week after each immunization and ELISA was performed to measure the binding of sera to recombinant BARF1 protein (FIGS. 2B and 2C). pBARF1 elicited a rapid seroconversion on day21, one week after the second immunization. BARF1 binding was slightly higher in C57BL/6 mice than BALB/c mice. The endpoint titers reached more than 1×10⁵ in both strains of mice after three immunizations (FIG. 2D).

pBARF1 Induces Potent Antigen-Specific and Polyfunctional T Cell Responses

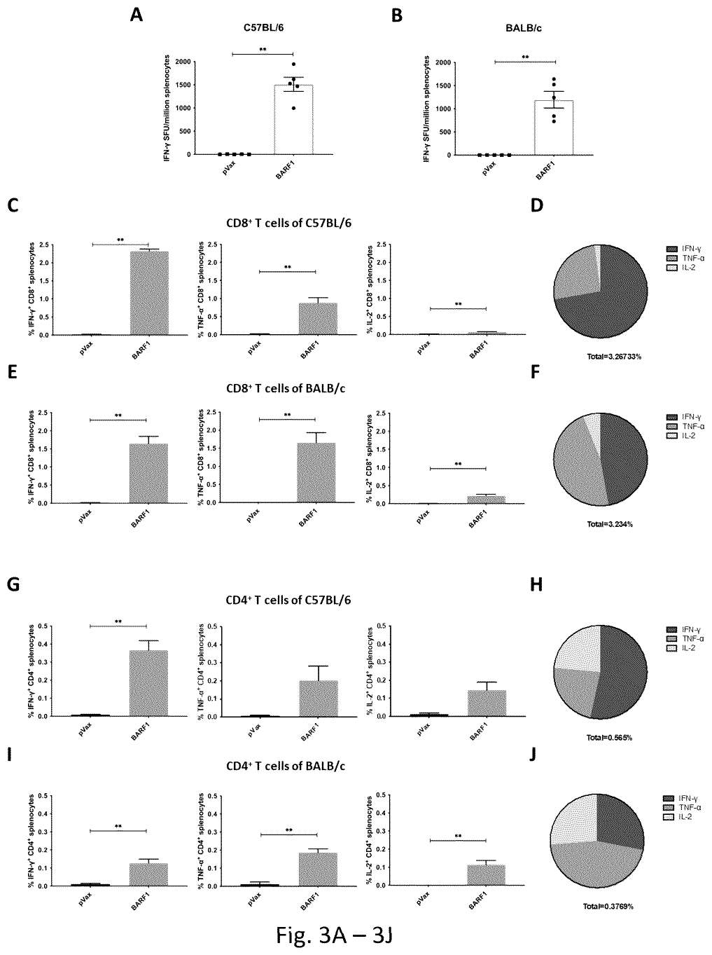

To evaluate the T cell response generated by pBARF1, mice splenocytes were harvested following the last immunization (FIG. 2A). Splenocytes were stimulated with BARF1 native peptides and evaluated by IFN-γ ELISpot assay. Significant activation of BARF1-specific IFN-γ T cell responses was observed in the pBARF1 group, with over 1000 spot forming units (SFU) in both C57BL/6 and BALB/c mice (FIGS. 3A and 3B). The intracellular cytokine production of stimulated splenocytes was analyzed by flow cytometry to study T cell phenotype (FIG. 3C-3J). Significant increases in IFN-γ, TNF-α, and IL-2 production was observed in CD8⁺ T cells from the pBARF1 immunized animals accounting for more than 3% of total CD3⁺ T cells. IFN-γ response was more pronounced in CD8⁺ T cells in C57BL/6 mice, while TNF-α and IFN-γ responses were activated similarly in BALB/c mice (FIG. 3C-3F). CD4⁺ T cells were activated in the pBARF1 group, but to a lesser extent than CD8⁺ T cells (FIG. 3G-3J). IFN-γ production by CD4⁺ T cells was elevated in both C57BL/6 and BALB/c mice. These results demonstrate that pBARF1 induces potent antigen-specific humoral and T cell responses in both C57BL/6 and BALB/c mice.

pBARF1 Improves Mice Survival in BARF1⁺ Carcinoma Models

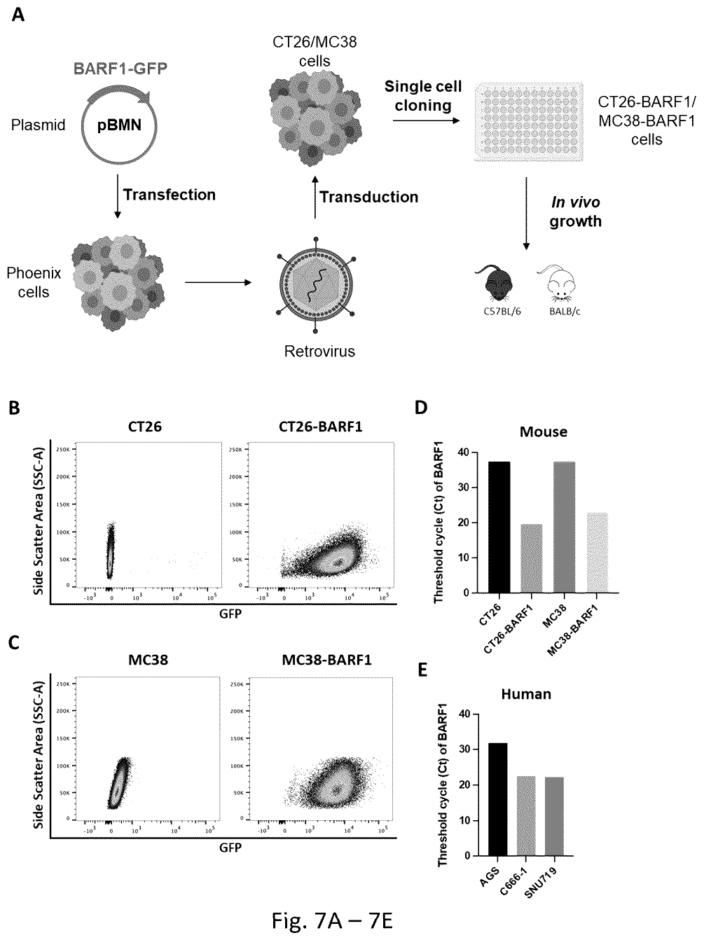

Next the impact of the immunity generated by immunization with pBARF1 was evaluated in murine tumor models. However, there are no EBV⁺ mouse cancer cell lines available for challenge as EBV does not infect mice. Therefore, two tumor models for these studies were generated by stably expressing BARF1 in carcinoma cell lines (FIG. 7A). A retroviral vector was used (pBMN-I-GFP) into which the BARF1 open reading frame (ORF) was cloned, and which also expresses GFP allowing one to follow transduction. The new vector (pBMN-I-BARF1-GFP) was used to generate retrovirus for transducing BARF1-GFP into MC38 and CT26 cells, which are murine colon adenocarcinoma cells syngenetic for C57BL/6 and BALB/c mice, respectively. Next single-cell cloning was performed on transduced cell lines to isolate stable clonal populations for additional study. Using flow cytometry, it was confirmed that there was a single GFP⁺ population of CT26-BARF1 or MC38-BARF1 cells, as validation of the clonality of the created cell lines (FIGS. 7B and 7C). To confirm BARF1 expression level in transduced mouse cell lines and compare the levels of expression observed with human EBV⁺ cancer cells, mRNA levels of BARF1 were quantified by RT-qPCR. A panel of EBV positive and negative human cell lines were also studied side by side in this assay, including C666-1 (nasopharyngeal carcinoma; EBV⁺), SUN-719 (gastric tubular adenocarcinoma; EBV⁺), and AGS (gastric adenocarcinoma; EBV⁻). BARF1 transcripts in CT26-BARF1 and MC38-BARF1 cells (FIG. 7D), as well as C666-1 and SUN-719 cells (FIG. 7E), were detected at around 20 threshold cycles (Ct), suggesting a positive expression. The negative controls, CT26, MC38, and AGS cells, all showed amplification of BARF1 above 30 Ct, indicating background levels of BARF1 expression. These data confirmed the expression of BARF1 in the mouse cell lines following transduction and show that the levels of expression are within the range of human EBV⁺ cancer cells, allowing for one to study aspects of immune potency targeting these model cells.

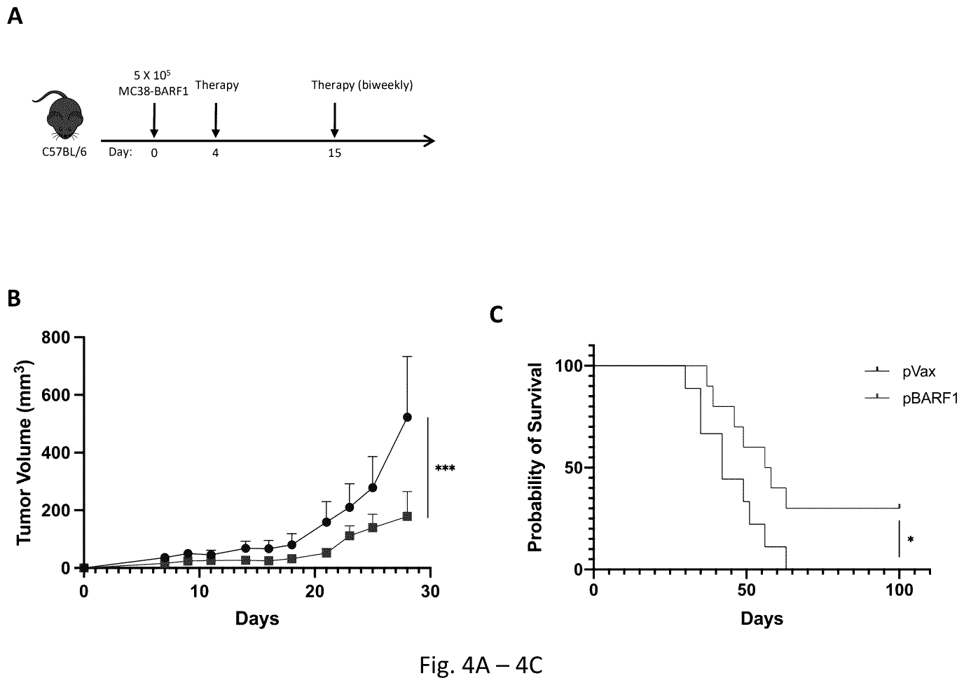

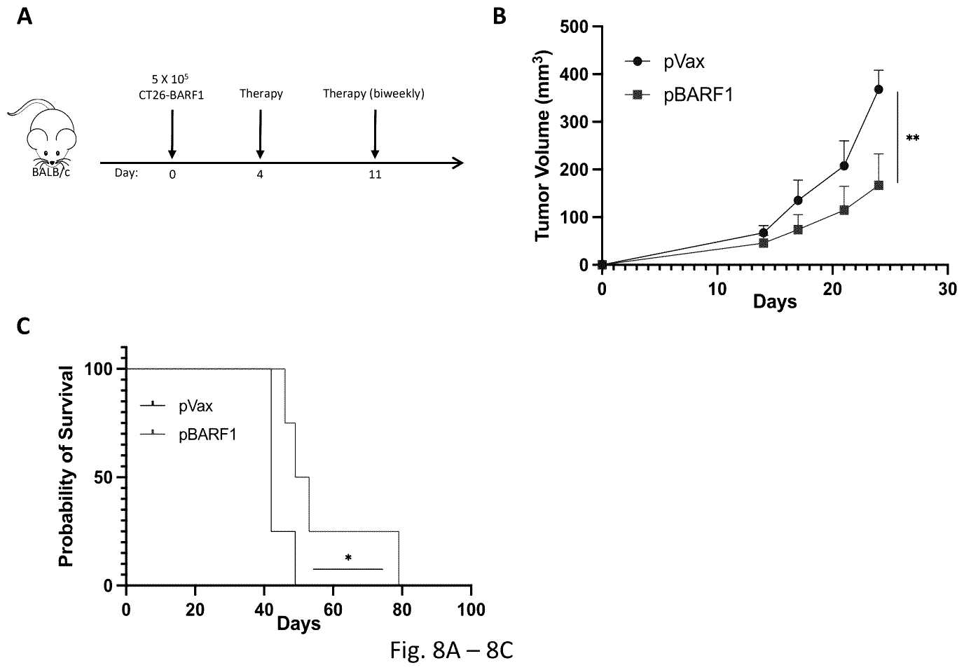

With these novel BARF1⁺ carcinoma models, next the therapeutic efficacy of pBARF1 was studied. 5×10⁵ MC38-BARF1 cells were injected subcutaneously on the flank of C57BL/6 mice and they were subsequently immunized with pBARF1 or pVax on day 4 and every two weeks following tumor challenge (FIG. 4A). Tumor growth was followed over time and a significant decrease of tumor burden in the pBARF1-immunized mice was observed compared to the pVax immunized animals (FIG. 4B). At the end of the study, the survival of pBARF1 treated animals was significantly improved over the control animals (FIG. 4C), with 3 out of 10 animals in the pBARF1 group appearing to completely control cancer for 100 days. Additionally, similar therapeutic efficacy of pBARF1 was observed in BALB/c mice challenged with the CT26-BARF1 cells, but without complete tumor control (FIG. 8). Overall, pBARF1 demonstrates a significant impact on treating established BARF1⁺ carcinomas.

Single Immunization of pBARF1 Completely Suppresses Tumor Growth in a CD8⁺ T Cell-Dependent Manner

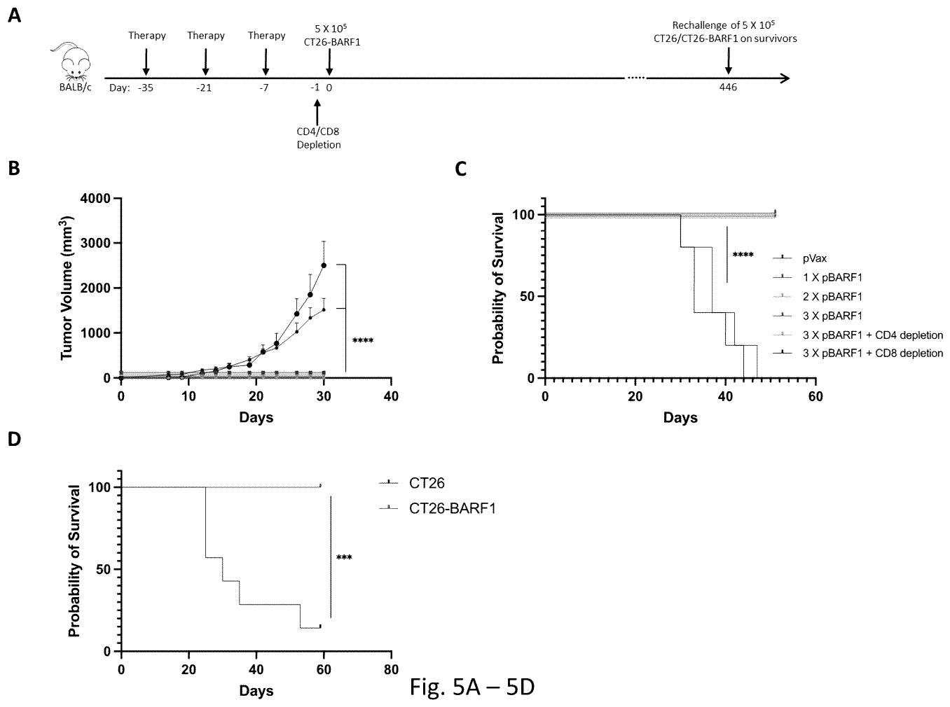

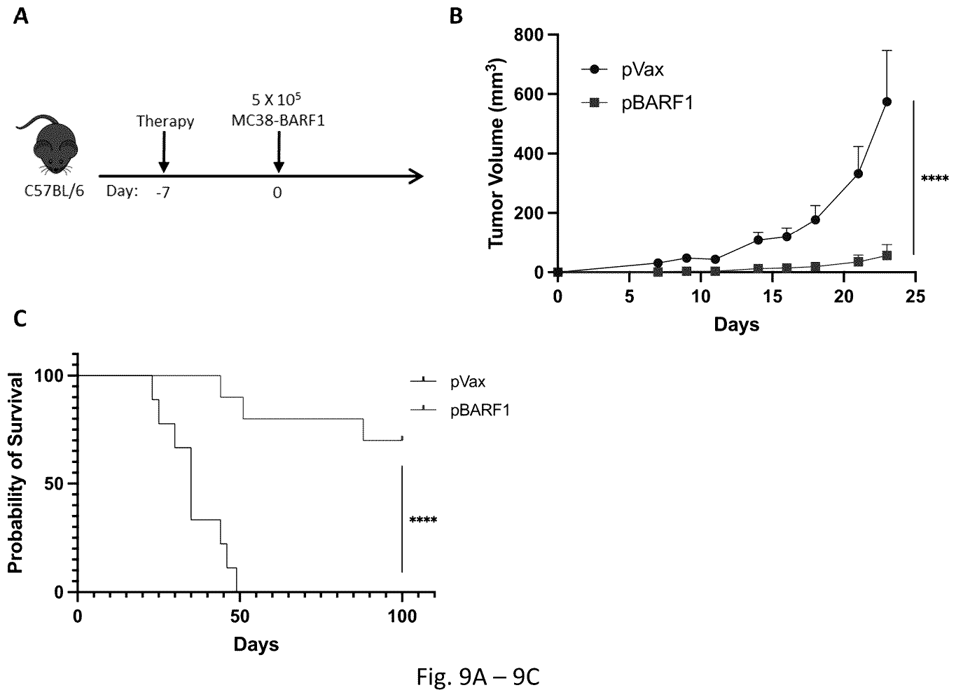

Next, the efficacy of pBARF1 in a pre-challenge immunization model was evaluated. BALB/c mice were immunized with one, two, or three doses of pBARF1 or pVax one week before tumor inoculation (FIG. 5A). To explore the contribution of different components of the immune response to tumor rejection, CD4⁺ or CD8⁺ T cells were also depleted starting one day before tumor challenge in additional pBARF1 immunized groups. Mice receiving one, two, or three doses of pBARF1, completely suppressed tumor growth, while the pVax group exhibited significantly higher tumor volume (FIG. 5B). CD4⁺ T cell depletion in mice immunized with pBARF1 did not affect tumor control. However, mice with CD8⁺ T cell depletion completely lost tumor control and exhibited a high tumor burden similar to the controls (FIG. 5B), suggesting CD8⁺ T cells are important for the therapeutic efficacy of pBARF1. Immunization one, two, or three times with pBARF1 resulted in 100% survival rates following the challenge (FIG. 5C). Furthermore, to evaluate the memory immune responses, the mice that received one, two, or three doses of pBARF1 and had survived tumor challenge were randomized and rechallenged with either CT26 or CT26-BARF1 cells on day 446 post initial challenge (FIGS. 5A and 5C). These mice only rejected CT26-BARF1 but not native CT26 cells, indicating that long-term BARF1-specific immunity was induced (FIG. 5D). Additionally, protection after a single immunization with pBARF1 was also observed when studied in the MC38-BARF1 model (FIG. 9).

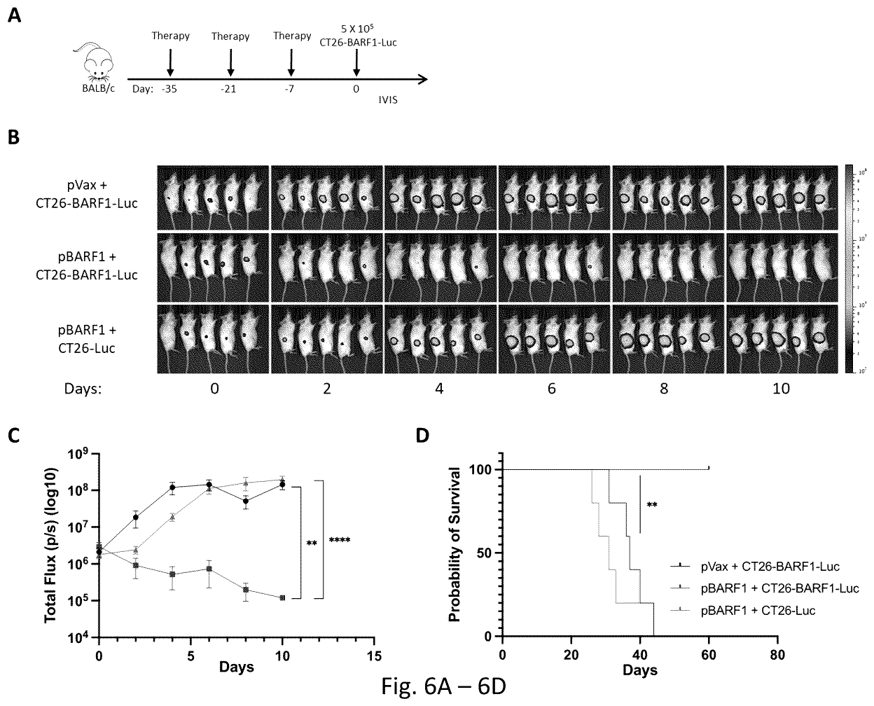

Finally, to study tumor clearance over time, animals were immunized and then tumor growth was monitored in vivo by using an IVIS imaging system. For this study, CT26-BARF1 or CT26 cells were transduced with CMV-Firefly luciferase lentivirus and CT26-BARF1-Luc or CT26-Luc cell lines were developed for challenge studies. BALB/c mice were immunized with three doses of either pBARF1 or pVax control plasmid and both groups were challenged with the CT26-BARF1-Luc or CT26-Luc cells (FIG. 6A). Mice immunized with pBARF1 showed clearance of CT26-BARF1-Luc as early as two days post-challenge (FIG. 6B). By day 10, all animals exhibited no detectable tumor burden, and tumor-free survival was observed (FIGS. 6C and 6D). In contrast, no control and rapid tumor growth was observed in the pVax group. The second control group, in which mice were immunized with pBARF1 and challenged with CT26-Luc, also showed cancer progression (FIG. 6B-6D). Together these data indicate pBARF1 mediated T cell immunity was focused on BARF1 displayed on the tumor cell and not an irrelevant cell target, supporting the specificity of the anti-tumor response.

### DISCUSSION

Here is provided the first study of the immune impact of BARF1 in a mouse model. It was shown that immunization with a DNA vaccine encoding BARF1 can drive antigen-specific immunity against BARF1 and impact cancer progression in a model system. Successful challenge outcome appears to be mostly dependent on the CD8⁺ T cell response which was associated with both short and long-term protection. Using a pre-challenge immunization model, it was shown that one dose of pBARF1 completely suppressed cancer progression. These results provide supportive evidence that immunity to BARF1 may be capable of targeting EBV cells that express BARF1 for immune clearance which has important implications for possible immune therapy of EBV-driven cancers.

Structural studies have shown that BARF1 can form as a hexamer, which acts as a decoy receptor for human M-CSF (Shim, A. H., et al., 2012; Proc. Natl. Acad. Sci. U.S.A. 109, 12962-12967). BARF1 interferes with M-CSF and receptor binding, and this interaction disturbs monocytes differentiation, which potentially affects macrophage polarization in the tumor microenvironment (TME). However, there is limited knowledge regarding how BARF1 affects TME status in NPC or EBVaGC. Here, potent humoral responses were induced by pBARF1, suggesting strong immune reactivity (FIG. 2). However, antibody responses were not sufficient to protect mice from tumor challenge, as CD8⁺ T cell depletion completely reversed the therapeutic effects of pBARF1 (FIG. 5A-5C). As BARF1 was reported not to interfere with differentiation of mouse monocytes (Shim, A. H., et al., 2012; Proc. Natl. Acad. Sci. U.S.A 109, 12962-12967), the potential therapeutic effect of humoral responses against BARF1 and its impact on TME need to be further investigated in a more relevant model that can assess the impact of the serology induced on M-CSF-BARF1 interactions. The development of such a model is important.

Many studies describe the importance of CTL responses for cancer immunotherapy (Farhood, B., et al., 2019, J. Cell. Physiol. 234, 8509-8521). In regard to immune therapy with DNA, a phase Jib study testing VGX3100 for women with high-grade cervical dysplasia was previously reported. It was shown that DNA immunization induced potent HPV E6 and E7-specific CTL responses (Trimble, C. L., et al., 2015, The Lancet 386, 2078-2088). Functional T cell responses were also identified as important biomarkers for patient response (Morrow, M. P., et al., 2016, Mol Ther Oncolytics 3, 16025; Morrow, M. P., et al., 2018, Clin. Cancer Res. 24, 276-294). Similarly, it was observed that DNA immunotherapy (MEDI0457) induced tumor-infiltrating T cells in patients with HPV-associated advanced head and neck squamous cell cancer (HNSCCa), highlighting the ability of this approach to drive virally relevant CTL as a tool for immune therapy of a virally driven cancer (Aggarwal, C., et al., 2019, Clin. Cancer Res. 25, 110-124). Consistent with these studies, here, it was found that pBARF1 induced potent CTL responses in two strains of mice, and these T cells responses were correlated with clearance of MC38-BARF1 or CT26-BARF1 tumors (FIG. 5). The present model systems further highlight the importance of CD8⁺ T cell effector function (FIG. 3) in tumor control as we observed increased tumor control in C57BL/6 mice (FIG. 4), which had a CD8⁺ T cell cytokine profile dominated by IFN-γ secretion as compared to BALB/c mice which had slightly decreased tumor control (FIG. 8), and a CD8⁺ T cell cytokine profile dominated equally by both IFN-γ and TNF-α.

This therapeutic outcome is of relevance to human immunotherapy, as the examination of some EBV-infected patients showed that BARF1 induced both CD4⁺ and CD8⁺ T cell responses as evidenced in EBV seropositive patients (Martorelli, D., et al., 2008, Int. J. Cancer 123, 1100-1107; Kalra, M., et al., Cytotherapy 21, 212-223; Pasini, E., et al., 2009, Int. J. Cancer 125, 1358-1364). BARF1-specific CTL were shown to kill EBV+ cancer in vitro. However, these studies did not test if the CTLs induced could influence tumor growth in vivo. The present models are limited in that mouse T cells cannot recognize BARF1 antigen presented by human HLA molecules, direct killing of human NPC or EBVaGC cells by pBARF1-induced CTL was not able to be evaluated in this study. As a first pass in this regard, it was observed that the transduced and cloned mouse carcinoma cell lines developed, CT26-BARF1 and MC38-BARF1, expressed similar levels of BARF1 as compared to human NPC and EBVaGC cell lines, C666-1 and SUN719, which have been previously obtained from EBV⁺ cancer patients (FIGS. 7D and 7E). While more development is important, this data supports in part the physiological similarity between the present models and human EBV⁺ cancer.

DNA antigen immunogenicity has been enhanced by various strategies (Suschak, J. J., et al., 2017, Hum. Vaccin. Immunother. 13, 2837-2848). Here, codon and RNA optimization and adaptive electroporation delivery was adopted for pBARF1, to enhance expression and immunogenicity (Suschak, J. J., et al., 2017, Hum. Vaccin. Immunother. 13, 2837-2848; Smith, T. R. F., et al., 2020, Nat Commun 11, 2601). Other strategies to enhance DNA immunotherapy include formulation with adjuvants, such as IL-12, and nanoparticle assembly of the antigen, as we recently reported (Xu, Z., et al., 2020, Advanced Science; Hutnick, N. A., et al., 2011, Curr. Opin. Virol. 1, 233-240). For immunotherapy in humans additional important antigens as part of an immune cocktail may be important. Combining BARF1 with other EBV latent proteins, such as EBNA1, LMP1, and LMP2 should be considered for future immunotherapy studies (Taylor, G. S., et al., 2014, Clin. Cancer Res. 20, 5009-5022; Taylor, G. S., et al., 2004, J. Virol. 78, 768-778; Lin, M. C., et al., 2017, BMC Cancer 17, 18). This multi-target approach would cover EBV⁺ cancer cells at different latency phases with diverse protein expression levels, thus possibly providing an additional advantage for limiting the chance of tumor escape.

Combined immunotherapy has been investigated in both preclinical and clinical studies. Pembrolizumab, a checkpoint inhibitor against PD1, was approved for recurrent and metastatic nasopharyngeal carcinoma, but the overall response rate is only 26.3% (Hsu, C., et al., 2017, J. Clin. Oncol. 35, 4050-4056).¹⁰ The non-responders are likely to be patients with low tumor-infiltrating lymphocytes (TILs). Although the immunosuppressive tumor microenvironment can be reshaped by anti-PD1, the CTL might not be abundant enough to control cancer cells. Combining pBARF1 with checkpoint inhibitors may support TIL abundance and enhance tumor clearance synergistically (Duperret, E. K., et al., 2018, Mol. Ther. 26, 435-445; Karyampudi, L., et al., 2014, Cancer Res. 74, 2974-2985; Soares, K. C., et al., 2015, J. Immunother. 38, 1-11).

In conclusion, herein is provided evidence in a mouse model of the relevance of BARF1 in immunotherapy for EBV-driven cancer. The immune potency of these vaccinations was highly impactful. Further study of BARF1 and immunotherapy for EBV is important and may represent a new tool to expand treatment options for patients with EBV-associated cancer.

### Materials and Methods

### DNA Plasmids

The pBARF1 plasmid construct was designed by adding a Kozak sequence and an immunoglobulin E (IgE) leader sequence to the N terminus of the native BARF1 sequence (amino acid 21-221, Uniprot: P03228). It was codon and RNA optimized and cloned into the modified pVax vector between restriction site EcoRI and Notl (Genscript). For the plasmid of pBMN-I-BARF1-GFP, native BARF1 sequence was codon and RNA optimized and inserted (Genscript) into a retroviral vector, pBMN-I-GFP (Nolan Lab; Addgene plasmid #1736).

### Cell Lines, Transfection, and Transduction

CT26, MC38, HEK293T, Phoenix, and AGS cells were obtained from the ATCC. SNU-719 was purchased from Korean Cell Line Bank. C666-1 was provided by Dr. Paul Lieberman at the Wistar Institute. For in vitro transfection, Lipofectamine 3000 (Invitrogen) was used following the manufacturer's instructions. For transduction of BARF1 into CT26 and MC38, the retrovirus was produced in Phoenix cells by transfecting with pBMN-I-BARF1-GFP and added to CT26 and MC38 cells. Single-cell cloning by limiting dilution was used to select GFP⁺ clones of transduced CT26 and MC38 cells. For transduction of luciferase into CT26-BARF1 and MC38-BARF1, a CMV-Firefly luciferase lentivirus (Cellomics Technology) was used following the manufacturer's instructions. All cell lines were maintained in RPMI1640 with 10% FBS and 1% penicillin and streptomycin (R10). They were routinely tested for Mycoplasma contamination.

### Immunoblotting

Recombinant BARF1 protein was synthesized by Genscript. Cell lysis, protein extraction, denaturation, and western blotting were done as previously described (Xu, Z., et al., 2020, Advanced Science). PVDF membranes were blotted with mouse anti-BARF1 serum as the primary antibody and goat anti-mouse IgG-HRP (ab6789, Abcam) as the secondary antibody. The signal was developed by SignalFire ECL reagent (Cell Signaling Technology), and images were captured by Amersham Imager 680 (GE Healthcare Life Sciences).

### Reverse Transcription and Quantitative PCR

Total RNA was isolated from cell lines by Rneasy Mini Kit (Qiagen), and cDNA was synthesized using high-Capacity cDNA Reverse Transcription Kit (Applied Biosystems), following the manufacturer's instructions. The mRNA expression of BARF1 was determined by quantitative PCR, using the Power SYBR Green Master Mix (Applied Biosystems) and QuantStudio 5 PCR System (Applied Biosystems). Primers were synthesized by Integrated DNA Technologies: transduced BARF1, 5′-CTTCATCGAGTGGCCCTTT-3′ (forward) (SEQ ID NO:9) and 5′-CTTCATCCTGCACAGGTAGTT-3′ (reverse) (SEQ ID NO:10); native BARF1 5′-GCCTCTAACGCTGTCTGTCC-3′ (forward) (SEQ ID NO:11) and 5′-GAGAGGCTCCCATCCTTTTC-3′ (reverse) (SEQ ID NO:12) (Hoebe, E., et al., Cancers (Basel) 10).

### Animal Immunization

C57BL/6 and BALB/c mice were purchased from The Jackson Laboratory. 25 μg of DNA plasmid (pBARF1 or pVax) in 30 μL water was injected into the tibialis anterior (TA) muscle, followed by delivery of two 0.1 Amp electric constant currents square-wave pulses by the CELECTRA-3P device (Inovio Pharmaceuticals). The immunization schedule is indicated in each figure. All procedures were done under the guidelines of the Wistar Institute Animal Care and Use Committee.

### Tumor Studies

C57BL/6 and BALB/c mice were immunized as described in the previous section at multiple doses before or after tumor challenge, as illustrated in each figure. 5×10⁵ of CT26, CT26-BARF1, MC38, MC38-BARF1, CT26-Luc, or CT26-BARF1-Luc cells (all under five passages) were injected subcutaneously into the right flank of the animals. Tumors were measured three times a week by electric calipers, and tumor volume was calculated by the formula: volume=0.5×height×width². Mice were euthanized when any dimension of the tumor reached 20 mm. For depletion of CD4⁺ and CD8⁺ T cell, 200 g of anti-CD8a (YTS169.4, BioXCell) and anti-CD4 (GK1.5, BioXCell) antibodies were injected intraperitoneally to each mouse twice a week until the end of the study. For the in vivo imaging system (IVIS) study, 200 μL of D-Luciferin (GoldBio) was injected intraperitoneally into each mouse, and bioluminescence signal was captured by IVIS SpectrumCT (PerkinElmer).

### ELISpot Assay

Spleens from immunized mice were harvested and dissociated by a stomacher. Red blood cells were removed by ACK lysing buffer. The splenocytes were filtered and counted. 2×10⁵ splenocytes were plated into each well on Mouse IFN-γ ELISpot^{PLUS} plates (Mabtech) and stimulated for 20 hours with BARF1 peptides (15mer peptides overlapping by 9 amino acid from the native BARF1, Genscript). Cells were stimulated with 5 μg/mL of each peptide in complete media (R10). The spots were developed based on the manufacturer's instructions. R10 and cell stimulation cocktails (Invitrogen) were used for negative and positive controls, respectively. Spots were scanned and quantified by ImmunoSpot CTL reader. Spot-forming unit (SFU) per million cells was calculated by subtracting the negative control wells.

### Intracellular Cytokine Staining and Flow Cytometry

Splenocytes were stimulated by BARF1 peptides for 5 hours with a protein transport inhibitor (Invitrogen). Cell stimulation cocktail and R10, with protein transport inhibitor, were used as positive and negative controls, respectively. After stimulation, cells were stained with LIVE/DEAD violet for viability. CD3e (17A2), CD4 (RM4-5), CD8b (YTS156.7.7), IFN-γ (XMG1.2), TNF-α(MP6-XT22), and IL-2 (JES56-5H4) fluorochrome-conjugated antibodies (all from BioLegend) were used for surface and intracellular staining. The samples were run on an 18-color LSRII flow cytometer (BD Biosciences) and analyzed by FlowJo software.

### Binding ELISA

NUNC MaxiSorp 96-well plates (Thermo Scientific) were coated with 1 μg/mL recombinant BARF1 (Genscript) in PBS overnight at 4° C. The plates were washed with PBS-0.5% Tween 20 and blocked with PBS-10% fetal bovine serum. Next, the plates were incubated with diluted mouse sera for two hours and goat anti-mouse IgG-HRP (Abcam) for one hour at room temperature. TMB (Thermo) was used to develop the binding signal.

### Statistical Analysis

All statistics were analyzed using GraphPad Prism 9. Error bars represent mean±SEM. For differences between the means of groups, significance was determined by the nonparametric Mann-Whitney Utest. For mouse tumor volume measurements, significance was determined by two-way ANOVA. For mouse survival studies, significance was determined by the log-rank test.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An immunogenic composition comprising one or more Epstein Barr Virus (EBV) antigenic polypeptide.

2. The immunogenic composition of claim 1, wherein said one or more EBV antigenic polypeptide comprises BamHI-A rightward frame 1 (BARF1) polypeptide.

3. The immunogenic composition of claim 1, wherein said one or more EBV antigenic polypeptide comprises a signal peptide.

4. The immunogenic composition of claim 3, wherein said signal peptide comprises an IgE signal peptide.

5. The immunogenic composition of claim 2, wherein said BARF1 polypeptide comprises one or more selected from the group consisting of:
a. a polypeptide comprising an amino acid sequence at least 90% identical to SEQ ID NO: 7;
b. a polypeptide fragment comprising an amino acid sequence at least 90% of the full length of SEQ ID NO: 7; and
c. a polypeptide fragment comprising an amino acid sequence at least 90% identical to an amino acid sequence at least 90% of the full length of SEQ ID NO: 7.

6. The immunogenic composition of claim 2, wherein said BARF1 polypeptide comprises the amino acid sequence of SEQ ID NO: 7.

7. An immunogenic composition comprising a nucleic acid molecule encoding one or more Epstein Barr Virus (EBV) antigenic polypeptide.

8. The immunogenic composition of claim 7, wherein said nucleic acid molecule comprises a nucleotide sequence encoding BamHI-A rightward frame 1 (BARF1) polypeptide.

9. The immunogenic composition of claim 7, wherein said nucleic acid molecule comprises a nucleotide sequence encoding a signal peptide.

10. The immunogenic composition of claim 9, wherein said nucleic acid molecule comprises a nucleotide sequence encoding an IgE signal peptide.

11. The immunogenic composition of claim 8, wherein said nucleic acid molecule comprises one or more selected from the group consisting of:
a. a nucleic acid molecule comprising a nucleotide sequence at least 90% identical to SEQ ID NO: 8;
b. a nucleic acid fragment comprising a nucleotide sequence at least 90% of the full length of SEQ ID NO: 8; and
c. a nucleic acid fragment comprising a nucleotide sequence at least 90% identical to a nucleotide sequence at least 90% of the full length of SEQ ID NO: 8.

12. The immunogenic composition of claim 8, wherein said nucleic acid molecule comprises the nucleotide sequence of SEQ ID NO: 8.

13. The immunogenic composition of claim 8, wherein said nucleic acid molecule comprises a codon and RNA optimized nucleotide sequence for expression in mammalian cells.

14. The immunogenic composition of claim 8, wherein said nucleic acid molecule comprises a plasmid expression vector.

15. The immunogenic composition of claim 14, wherein said plasmid expression vector comprises a pVax expression vector.

16. A method of administering an immunogenic composition to a subject, comprising administering to the subject one or more selected from the group consisting of:
a. a composition comprising a nucleic acid molecule comprising a nucleotide sequence at least 90% identical to SEQ ID NO: 8; and
b. a composition comprising a polypeptide comprising an amino acid sequence at least 90% identical to SEQ ID NO: 7.

17. A method of inducing an immune response to one or more EBV antigen in a subject, comprising administering to the subject an immunogenic composition according to the method of claim 16.

18. A method of treating or preventing one or more disease or disorder associated with EBV infection in a subject in need thereof, comprising administering to the subject an immunogenic composition according to the method of claim 16.

19. The method of claim 18, wherein said disease or disorder is cancer.

20. The method of claim 19, wherein said cancer comprises one or more selected from the group consisting of: nasopharyngeal carcinoma (NPC), EBV-associated gastric carcinoma (EBVaGC), Hodgkin's lymphoma, Burkitt lymphoma, Diffuse large B cell lymphoma, T cell lymphoma, and NK cell lymphoma.
